(19) United States
(12) Patent Application Publication
(10) Pub. No.: US 2004/0248128 A1
(43) Pub. Date: Dec. 9, 2004

(54) Method of analyzing interaction between material and biological substance

(76) Inventors: Sakura Sakano, Shizuoka (JP); Ayako Isoai, Shizuoka (JP)

Correspondence Address: BIRCH STEWART KOLASCH & BIRCH, PO BOX 747, FALLS CHURCH, VA, 22040-0747, US

(21) Appl. No.: 10/487,905

(22) PCT Filed: Aug. 27, 2002
(86) PCT No.: PCT/JP02/08614

(30) Foreign Application Priority Data
Aug. 27, 2001 (JP) 2001-256425
Jan. 15, 2002 (JP) 2002-6547
Jan. 16, 2002 (JP) 2002-6859

(51) Int. Cl. C12Q 1/68; G01N 33/53; G01N 33/567

(52) U.S. Cl. 435/6; 435/7.2

(57) ABSTRACT

The present invention provides a method of analyzing an interaction between a medical material and a biological substance. The method includes bringing particles having a material to be evaluated at least on the surface thereof into contact with a biological substance to thereby form composite particles, binding the biological substance directly or indirectly to a fluorescent label, and then measuring the fluorescence intensity of the bound matter with a flow cytometer to thereby analyze the interaction between the material and the biological substance. Based on the extent of this interaction, it can be assessed whether or not the material is usable as a medical material for artificial vessels and so on.

### TECHNICAL FIELD

The present invention relates to a method for analyzing an interaction between materials such as synthetic polymers and biological substances such as plasma proteins, and to a medical material selected by using the method.

The analysis method of the present invention can be used for selecting a material that is most suitable for various medical instruments such as hemodialyzers, leukocyte-removing filters for transfusion, or artificial blood vessels. The medical material of the present invention can be used for manufacturing various medical instruments such as hemodialyzers, leukocyte-removing filters for transfusion, and artificial blood vessels.

### BACKGROUND ART

Various medical instruments such as hemodialyzers, leukocyte-removing filters for transfusion, and artificial blood vessels are composed of so-called biocompatible materials. Properties required for the biocompatible materials differ from each other. For example, hollow fibers that are the main part of a hemodialyzer are preferably composed of a material that does not adsorb plasma proteins or cells (leukocytes, erythrocytes, or platelets) in blood, while a filter material that is the main part of a leukocyte-removing filter for a platelet product is desirably composed of a material that adsorbs leukocytes but does not adsorb platelets. Recently, the properties required for the respective materials become more rigorous, and researches and developments for novel materials have been performed extensively. When the materials are designed and made on an experimental basis (typically, not one material but many materials are simultaneously designed and made on an experimental basis), the properties of the materials are firstly evaluated in a simple in vitro system for selection (screening) to progress to the next stage (for example, under conditions more similar to that occurring in practical use, in which the materials are molded into the same forms as those in practical use and are assembled into an appliance). The evaluation in the simple in vitro system is typically performed by evaluating the adsorption of plasma proteins or platelets to a plate coated with the material. However, this method has a problem in that the surface area for adsorption is small and stress such as three-dimensional shear stress is difficult to apply to such an adsorption reaction system. Ishihara et al. (Polymer Chemistry 34, 188-205, 1996) analyzed the adsorption amount of fibrinogen indirectly by processing the material into a particle form and monitoring decrease in UV absorption at 230 nm. Thus, the surface area for adsorption can be enlarged by processing the material into a particle form compared with the surface when using a plate. However, in the indirect measurement of change in the protein concentration, evaluation can be performed only with a single index and there is difficulty in evaluating many materials simultaneously with many indices.

At present, “flow cytometers”, which have been developed and increasingly used in Europe and America in 1970s, are analysis apparatus essential not only in fundamental science fields including biology and immunology but also in clinical examination fields including diagnosis for leukemia. The term “flow cytometer” is a generic term for an apparatus for analyzing physical or biological properties of cells by optical or electrical signals obtained by suspending the cells (or cell components such as chromosomes and the like) in a solution, and passing the cells into a fluid system at high speed, through a detection portion provided within the fluid system (Shigeru Takamoto, published by Igaku-shoin Ltd., “Applied Cytometry”, p. 20).

As described above, flow cytometers are very widely applied for analyzing cells (or cell components). However, a method in which a flow cytometer is used for evaluating the material by passing the material instead of cells is a completely different technical idea, which cannot be conceived by a person skilled in the art at all. JP 11-83724 A discloses a method in which spherical particles that imitate cells and are composed of a synthetic polymer having a specific particle size and a specific monodispersibility, are passed through a flow cytometer. However, the purpose of the method is to use the spherical particles composed of a synthetic polymer for calibration of the flow cytometer, which is completely different from the present invention in which the particles are used for screening a biocompatible material. In addition, WO 99/64867 discloses use of a flow cytometer for selecting a physiologically active substance to be used for a drug. However, a physiologically active substance such as a drug having a low molecular weight is completely different from a medical material, which is the desired target of to the present invention, in the function, properties, and structure.

### DISCLOSURE OF THE INVENTION

An object of the present invention is to provide a method in which an interaction between a material and a biological substance can be analyzed easily and rapidly by using many materials simultaneously with many indices based on the intended use of the material under various environments.

The inventors of the present invention have made extensive studies in order to solve such problems. As a result, they have found a means for attaining an object of the present invention by making use of the aforementioned flow cytometer that have been previously used in a completely different field from that of evaluation of a material. That is, the following hypothesis was conceived and proved to accomplish the present invention: various evaluations similar to those of cells can be performed by processing a material to be evaluated into the form of spherical particles, similar to that of cells previously used, bringing the particles into contact with a biological substance to be evaluated, and passing the spherical particles through a flow cytometer instead of cells.

The present invention relates to a method for analyzing an interaction between a material and a biological substance using a flow cytometer. In addition, the present invention relates to a medical material selected by using the analysis method.

Specifically, the present invention relates to a method of analyzing an interaction between particles having a material to be evaluated at least on the surface thereof and a biological substance by using a flow cytometer.

More specifically, the term “interaction between particles having a material to be evaluated at least on the surface thereof and a biological substance” refers to a reaction performed by bringing the particles into contact with the substance, and is a method of analyzing biological substance composite particles prepared by the reaction. The biological substance composite particles can be analyzed by fluorescent labeling of the biological substance directly or indirectly.

The materials to be evaluated in the present invention include all medical materials except drugs.

Moreover, the present invention relates to a medical material selected by using the aforementioned analysis method.

In addition, the present invention relates to a medical material selected from a plurality of medical materials to be evaluated, which satisfies a predetermined criterion of an interaction between a biological substance and the material.

The particles having a material to be evaluated at least on the surface thereof can be produced by coating the surface of particles composed of a base material with a polymer material by physical adsorption. The properties of the surfaces of the thus-prepared polymer-coated particles are assessed as follows. The particle surfaces are judged to have the properties of the material uniformly in the case where an element composition ratio in the surface is substantially equal to that of the polymer material when inspected by X-ray photoelectron spectroscopy, or in the case where a mass peak intensity derived from an element of a base material particle is 10% or less than that derived from the polymer material when inspected by time-of-flight secondary ion mass spectrometry. The particles can be dispersed in a dispersion media. The base particles of the particles has a particle size of 0.1 μm or more and 100 μm or less, and have a sphericity of 0.50 or more. The polymer material to be coated has an average molecular weight of 1,000 or more and 3,000,000 or less. The steps of producing the particles include the steps of: suspending particles composed of a base material in a solution obtained by dissolving a polymer material in a solvent; applying physical vibration to the suspension; washing the particles with a washing solution that is a poor solvent for the polymer material and the base particles; and vacuum drying or freeze-drying the particles.

Alternatively, the particles having a material to be evaluated at least on the surface thereof can be produced by binding synthetic polymer construct base material particles having a polymer with a side chain capable of changing a functional group at least on the surface thereof to a functional group of a compound having a functional group via the side chain. Specifically, the particles can be produced by binding synthetic polymer construct base material particles having an acrylic or methacrylic polymer with a side chain capable of changing a functional group at least on the surface thereof to a functional group of a compound having a functional group via the side chain.

### BRIEF DESCRIPTION OF THE DRAWINGS

FIG. 1 shows a distribution state of mass peaks derived from styrene with respect to a total ion image (left side), which is obtained by measuring the particle surface in Comparative Example 1 by time-of-flight secondary ion-mass spectrometry.

FIG. 2 shows a distribution state of mass peaks derived from styrene with respect to a total ion image (left side), which is obtained by measuring the particle surface in Example 1 by time-of-flight secondary ion mass spectrometry.

FIG. 3 shows a distribution state of mass peaks derived from HEMA with respect to a total ion image (left side) in Comparative Example 1.

FIG. 4 shows a distribution state of mass peaks derived from HEMA with respect to a total ion image (left side) in Example 1.

FIG. 5 is a histogram showing a result of an analysis for dispersibility in Comparative Example 1 using forward scattered light (FSC Hight) of a flow cytometer. “M1” indicates a region of monodispersed particles.

FIG. 6 is a histogram showing a result of an analysis for dispersibility in Example 1 using forward scattered light (FSC Hight) in a flow cytometer. “M1” indicates a region of monodispersed particles.

FIG. 7 is a set of figures obtained by analyzing material particles in Example 1 using a flow cytometer, and the upper most figure shows the result of a negative control. The individuals (a) to (d) show results in the cases of copolymers having DM wt % of 0, 3, 6, and 10% in respective materials. The ordinate indicates relative values of fibrinogen adsorption with respect to fluorescence intensities of Alexa Fluor 647, and the abscissa indicates relative values of IgG adsorption with respect to fluorescence intensities of FITC.

FIG. 8 is a histogram showing adsorption of particles in Comparative Example 2 and fibrinogen. The histogram is obtained as a result of detecting the adsorption of fibrinogen indirectly with an FITC-labeled goat anti-human fibrinogen antibody, and indicates fluorescence intensities of FITC. The region (M1) in the figure shows a positive region with respect to a negative control.

FIG. 9 is a histogram showing adsorption of the particles in Example 2 and fibrinogen. The histogram is obtained as a result of detecting the adsorption of fibrinogen indirectly with an FITC-labeled goat anti-human fibrinogen antibody, and indicates fluorescence intensities of FITC. The region (M1) in the figure shows a positive region with respect to a negative control.

FIG. 10 is a dot plot showing the binding state of fibrinogen and albumin in Comparative Example 2, which is measured by using a flow cytometer. The ordinate indicates fluorescence intensities of PE, which are indirectly detected by binding albumin adsorbed to particles to a biotin-labeled goat anti-human albumin antibody and further to PE-labeled avidin. The abscissa indicates fluorescence intensities of FITC, which are indirectly detected by binding fibrinogen adsorbed to particles to an FITC-labeled goat anti-human fibrinogen antibody. The lines in the figure indicate positive regions with respect to a negative control.

FIG. 11 is a dot plot showing a binding state of fibrinogen and albumin in Example 2, which is measured by using a flow cytometer. The ordinate indicates fluorescence intensities of PE, which are indirectly detected by binding albumin adsorbed to particles to a biotin-labeled goat anti-human albumin antibody and further to PE-labeled avidin. The abscissa indicates fluorescence intensities of FITC, which are indirectly detected by binding fibrinogen adsorbed to particles to an FITC-labeled goat anti-human albumin antibody. The lines in the figure indicate positive regions with respect to a negative control.

FIG. 12 is a set of figures obtained by analyzing platelet adsorption in Example 3 using a flow cytometer. The ordinate indicates fluorescence intensities of PerCP, which are obtained by detecting bonding of PerCP-labeled CD61. The abscissa indicates fluorescence intensities of FITC, which are obtained by detecting bonding of an FITC-labeled PAC-1 antibody. The upper left figure shows a result of a negative control. Parts (a) to (e) show a part of results of polymer particles obtained by polymerization of HEMA with DM or methyl methacrylate so as to have different wt %. Particles adsorbed to platelets are gated among monodispersed material particles in each figure. The numeral in each figure indicates the proportion of the particles.

FIG. 13 is a set of figures showing binding states of fibrinogen and IgG in Example 4, which are obtained by analysis using a flow cytometer. The ordinate indicates-fluorescence intensities of PE, which are indirectly detected by binding IgG adsorbed to particles to a biotin-labeled goat anti-human albumin antibody and further to PE-labeled avidin. The abscissa indicates fluorescence intensities of FITC, which are indirectly detected by binding fibrinogen adsorbed to particles to an FITC-labeled goat anti-human fibrinogen antibody. The upper left figure shows the result of a negative control. Individual figures (a) to (c) show results obtained by performing procedures in Example 4 using the particles prepared in Example 3. The lines in the figures indicate positive regions with respect to a negative control.

### BEST MODE FOR CARRYING OUT THE INVENTION

Hereinafter, the present invention will be described in more detail.

The term “flow cytometer” in the present invention refers to an apparatus for analyzing physical or biological properties of each cell by optical or electrical signals obtained by suspending cells in a solution, passing the cell into a fluid system at high speed, and passing the cell through a detection portion provided in the midway of the fluid system. Such an apparatus is commercially available from Becton, Dickinson and Company (BD) or Beckman Coulter, Inc. under the brand name of FACS or EPICS, respectively.

The term “material” in the present invention refers to one to be used in evaluation of an interaction with a biological substance for any given purpose, which may be any of a synthetic polymer compound, a natural polymer compound, an inorganic compound, and a metal. Examples of the synthetic polymer compound include copolymers containing polystyrene, polyethylene, polypropylene, polyester, polyurethane, polysulfone, polyhydroxyethyl methacrylate, polymethoxyethyl acrylate, and methacryloyl phosphorylcholine, and derivatives thereof, but there is no limitation thereto. Examples of the natural polymer compound include cellulose (including derivatives thereof), chitin, chitosan, and alginate, but there is no limitation thereto. In addition, examples of the inorganic compound include: ceramics such as titania, alumina, silica, and silicon nitride; and glasses composed of hydroxyapatite or tricalcium phosphate having various compositions, but there is no limitation thereto. Examples of the metal include stainless steel and titanium, but there is no limitation thereto. Also, the material in which an organic compound having a low molecular weight is used for modifying a metal or inorganic material surface is a target to be evaluated in the present invention.

Examples of such a material include materials to be used for many different medical instruments such as a hemodialyzer, a leukocyte-removing filter for transfusion an artificial blood vessel, and the like.

The term “biological substance” in the present invention refers to a subject substance when evaluating an interaction between the substance and the aforementioned material. Examples of the substance include: plasma proteins such as albumin, fibrinogen, and globulin; cytokines such as interleukin and interferon; growth factors such as insulin, HGF, and VEGF; extracellular matrixes such as laminin and collagen; and various cells such as platelets and leukocytes. The substance is appropriately selected according to the purpose. Specifically, for example, a plasma protein, a hemocyte, or the like is appropriately selected and used as a biological substance in the case where the substance is used for screening a material for a leukocyte-removing filter; or each of various biologically active substances is appropriately selected and used as a biological substance in the case where the substance is used for screening a base material in cell culture for regenerative medicine. Thus, the substance is appropriately selected according to the purpose.

The term “interaction” in the present invention mainly refers to adsorption of a biological substance to a material, which is used as an index in screening a material. Therefore, in the analysis of the interaction, the adsorption amount (percentage) is mainly measured. However, not only the adsorption amount (percentage) but also the structure of the biological substance and/or change in function caused by the adsorption can be also measured.

In the analysis method of the present invention, biological substance composite particles are prepared by bringing particles having a material to be evaluated at least on the surface thereof into contact with a biological substance for evaluating an interaction between the particles and the material.

In order to analyze the biological substance composite particles by using a flow cytometer, the biological substance is subjected to fluorescent labeling directly or indirectly. The fluorescent labeling of the biological substance itself can be performed by chemically binding the biological substance directly to the fluorescent dye, for example, an amine of a protein such as fluorescein isothiocyanate (FITC) that is one of the most general fluorescent dyes, Oregon Green 488 having a succinimidyl ester moiety (Molecular Probes, USA)., or Alexa Fluor 647 (Molecular Probes, USA), at a ratio of one or several dyes per protein molecule. In the case of indirect fluorescent labeling, the biological substance is subjected to fluorescent labeling via a substance having a specific affinity. Examples of the substance having the affinity include an antibody and lectin. The substance to be used may be one that has been subjected to fluorescent labeling previously, or one that has been labeled with biotin or no label. The substance having the affinity that has been labeled with biotin or no label is further subjected to second fluorescent labeling after the reaction with the biological substance composite particles of the present invention.

The particles having a material to be evaluated at least on the surface thereof is composed of either the material to be evaluated in the whole portion of the particles, or the material to be evaluated only on the surface thereof. The particles are prepared by using the material to be evaluated, or prepared so as to have the material to be evaluated on the surface by coating the particles provided as a core (the material of the particles may be either the same as or different from that to be evaluated) or by performing graft polymerization.

The particles having a material to be evaluated at least on the surface thereof in the present invention can be produced by coating the surface of particles composed of a base material with a polymer material by physical adsorption.

The base material in the present invention can be selected from any components. Specific examples thereof include: synthetic polymers such as polystyrene, polyethylene, polypropylene, acrylic resin, nylon, polyester, polycarbonate, polyacrylamide, polyurethane, and polyethylene terephthalate, and copolymers thereof; natural polymers such as agarose, cellulose, cellulose acetate, chitin, chitosan, and alginate; inorganic materials such as hydroxyapatite, silica, alumina, titania, and glass; and metals such as stainless steel, titanium, and aluminum.

The sphericity of the base material particles of the present invention is preferably 0.50 or more. The sphericity is represented by the following expression:

(sphericity)=(short diameter of particles)/(long diameter of particles),

and is particularly preferably 0.80 or more. In the case where the sphericity of polymer fine particles is less than 0.50, the particles are different in shape from one another and an energy distribution on the particle surface differs at respective portions. As a result, there is a fear that the particles could fail to be coated with a polymer material uniformly.

The particle size of the base material particles in the present invention is preferably 0.1 μm or more and 100 μm or less, more preferably 1 μor more and 50 μm or less.

The term “polymer material” in the present invention refers to a polymer to dissolve in any one of wide kinds of organic solvents such as water and ethanol, which has an average molecular weight of 1,000 or more and 3,000,000 or less. In the case where the material has a molecular weight of less than 1,000, the stability in adsorption of a polymer material for coating decreases, so that there may occur desorption from the base material particles. In the case where the material has a molecular weight of larger than 3,000,000, the viscosity of a polymer solution becomes higher, so that there may be caused difficulty in dispersion of the base material particles when being added to the polymer solution, or in preparation of particle surfaces having properties of the polymer material uniformly. Therefore, both cases are not preferred.

The term “physical adsorption” in the present invention refers to adsorption caused by a bond other than a chemical bond (formed by giving and receiving or covalence of an electron) or an interaction functioning between a base material surface and a polymer material. Specific examples thereof include bonds formed by any energy and interactions such as hydrophobic bonds, hydrogen bonds, dipole interaction, London dispersion force, electrostatic interaction, Van der Waals force, and charge transfer interaction.

Hereinafter, there will be explained in detail a method of producing polymer-coated particles, in which a polymer material is physically adsorbed uniformly to the surface of the base material particles in the present invention.

A solution of the polymer material is prepared so as to have a concentration resulting in no contact of polymer chains with each other in a solvent. Specifically, the solution is prepared so as to have a polymer concentration of 0.001 wt % or more and 20 wt % or less, preferably 0.01 wt % or more and 10 wt % or less. The type of the solvent should be one in which the base material particle is not dissolved and only the polymer material is dissolved. That is, it is necessary for the solvent to have properties of a poor solvent to the base material particles and have the properties of a good solvent to the polymer material to be used in coating. Examples of a poor solvent to base material particles composed of polystyrene include water, alcohols, ethers, and hydrocarbons. Examples of a good solvent to a polymer material composed of polyhydroxymethyl methacrylate or the like include alcohols, dimethylformamide, and dimethylsulfoxide. Therefore, for the polymer solution, alcohols are preferably selected. That is, any organic solvent can be used as-long as the solvent is suitable for solubility of the base material particles and the polymer material. The physical adsorption of the polymer to the base material particles is accomplished by suspending the base material particles in the thus-prepared polymer solution and by applying physical vibration to the suspension. Examples of the physical vibration include using ultrasound waves, stirring using a stirrer or the like, and using a shaker or a rotator. Although the effect of dispersing the particles in the polymer solution can be expected by any of the methods, the effect of dispersing aggregations formed of particles can further be expected in a method using ultrasound waves, and the effect of causing the polymer solution itself to flow can further be expected in a method using a stirrer, shaker, rotator, or the like. Therefore, both may be used in combination.

The time for applying the physical vibration may be any time as long as the time is 10 seconds or more. Preferred is a range between 1 minute and 48 hours. If the time is 10 seconds or less, there is a possibility that dispersibility of particles or mixing of the base material particle with the polymer solution is insufficient. The temperature to be applied in heating for an adsorption reaction is 0° C. or more and 200° C. or less, preferably 10° C. or more and 150° C. or less. If the temperature is higher than 200° C., there is a risk of difficulty in controlling the concentration of the polymer solution because the polymer material is decomposed and the solvent is evaporated. Further, if the temperature is lower than 0° C., there is a risk of occurrence of phase separation or precipitation of the polymer material. Separating particles from a polymer solution after the adsorption reaction can be performed by any method, including centrifugation, filtration, and the like.

A washing step for completely removing the polymer solution from a suspension of particles is needs to be performed in a liquid having properties as a poor solvent to both the base material particles and the polymer material. For example, for base material particles composed of polystyrene and for polyhydroxymethyl methacrylate provided as a polymer material, distilled water is desirably used as washing solution. After washing, the particles are dried under vacuum or freeze-dried, enabling stabilization of the material on the surface of the base material particles.

By surface analysis using X-ray photoelectron spectroscopy or time-of-flight secondary ion mass spectrometry, an element or a partial structure specific to the polymer material is detected. As a result, the polymer-coated particles of the present invention can be confirmed to uniformly have properties of the polymer material on the surface thereof. The term “uniformly” in the present invention refers to a state showing the properties of the polymer material evenly on the entire surface of the particles, preferably, a state in which the surface of the particle is entirely coated with the polymer material, but there is no limitation thereto.

X-ray photoelectron spectroscopy is a method of analyzing kinetic energy of photoelectrons released from the surface of a sample by irradiating the sample with monochromated X-rays. The method can provide qualitative and quantitative assessment of the composition of the elements existing on the surface of the sample at a depth of several tens of angstroms. In addition, information on the chemical state of each element can be obtained by appearance of, in the spectrum of each element, a chemical shift due to influence from the adjacent elements, a satellite considered to be based on charge transfer transition, split of core level due to bond of a multiplet, or the like. That is, the coating state of the polymer material can be confirmed by comparison of elements or partial structures specific to the base material particles and the polymer material in spectra obtained by the X-ray photoelectron spectroscopy. Comparison points for selection are preferably as follows: an element is contained in the base material particles but is not contained in the polymer material, and vice versa, and the element composition ratio of the base material particles in a common partial structure differs from that of the polymer material. When the polymer-coated particles are analyzed by X-ray photoelectron spectroscopy, it is shown that the particles have the properties of the polymer material uniformly because the resultant spectra and the element composition reflect the properties of the polymer material. However, it should be taken into consideration that the element composition obtained by the method is affected by differences in detection sensitivity depending on the element, the kind of X-ray source used, the release angle (depth to be measured) of photoelectrons released from a sample, or the like.

According to the report by K. B. Lewis et al. (J. Colloid Interface Sci., vol.159, 77, 1993), in the case where the element composition of a copolymer of styrene and hydroxyethyl methacrylate is determined for the release angle of photoelectrons in the aforementioned method, i.e., a depth to be analyzed, the copolymer has almost the same surface composition as that of the bulk material when the copolymer is a random copolymer, while the copolymer has a styrene-rich surface composition when the copolymer is a block copolymer. In addition, the spectrum in photoelectron spectroscopy is based on electron current intensity, i.e., count, so that there are statistical errors. Moreover, there is possibility that the signal intensity of the particles in the form of powder is less than that of a sample in the form of a plate; that the detection of photoelectrons is affected by the particles because the particles are generally charged to have a positive potential when a component thereof is an insulating substance; or the like. That is, the element composition ratio of the particle surfaces is substantially equal to that of the polymer material from the analysis by X-ray photoelectron spectroscopy. This means, in consideration of the fact that the element composition ratio of the particles may not always be equal to the theoretical element composition ratio based on molecules forming a polymer material, that when comparing the element composition of the polymer material with that of the particles both are almost identical.

Preferably, the element composition ratio obtained by measuring the polymer material to be used for coating particles under the same analysis condition is different from the element composition ratio of the particles by 15% or less, or the proportion of an element specific to the base material particles decreases to 10% or less, but there is no limitation thereto.

Time-of-flight secondary ion mass spectrometry is a method of performing mass analysis of many elements simultaneously by colliding the first ion with a sample to release an ion (secondary ion) from the sample, accelerating the ion in a magnetic field, and measuring the flight time of the ion. The sample surface can be analyzed from several angstroms to several tens of angstroms in depth, and it is suspected that about 90% or more of the obtained information is information derived from an atom layer from the outermost surface. Moreover, by observing the intensity distribution of mass peaks specific to each molecule as an image figure, a distribution state of a chemical structure on the material surface can be observed at submicron level. That is, a distribution state of a molecule on the outermost surface of polymer-coated particles can be evaluated by comparing mass peaks specific to the base material particles and the polymer material among resultant mass spectra or image figures showing intensity distributions of the peaks. Analysis of the polymer-coated particles by time-of-flight secondary ion mass spectrometry shows that the base material particles have properties of the polymer material uniformly when the mass peak intensity derived from an element composing the base material particles of the polymer-coated particles has a detection intensity of 10% or less than a mass peak intensity derived from an element of the base material particles.

Monodispersion of the polymer-coated particle in a dispersion medium can be measured by electrical impedance tomography or by using an analysis apparatus based on light scattering. For example, a flow cytometer can show that at least 80% of particles are monodispersed.

Methods of producing particles of the present invention having a material to be evaluated at least on their surfaces, other than the aforementioned methods include a method in which base material particles which are composed of a synthetic polymer construct and have an acrylic or methacrylic polymer having a side chain capable of changing a functional group at least on its surface, is bound to a functional group of a compound having a functional group via the side chain.

The term “side chain capable of changing a functional group” in the present invention refers to a side chain capable of changing a functional group variously by making to reacting with various molecules. Specific examples thereof include, in addition to a carboxyl group and an amino group, a side chain containing a reactive functional group such as hydroxyl, ammonium, ketone, a carboxylate ion, nitro, thiol, aldehyde, nitrile, isocyanate, or a heterocyclic compound such as epoxy.

As a polymer having the side chain capable of changing a functional group, there can be used a monopolymer having a side chain capable of changing a functional group, or a copolymer having at least one constitutional unit having at least a side chain capable of changing a functional group in the molecule.

Specific examples thereof include: a monopolymer composed of polyacrylic acid, polymethacrylic acid, acrylate, methacrylate, acrylic acid esters, methacrylic acid esters, acrylamides, methacrylamides, polyvinyl chloride, polyacrylonitrile, a styrene polymer, polyvinyl acetate, polyvinylpyridine, polyvinylpyrrolidone, polyethylene glycol, polyethylene oxide, hydrophilic polyurethane, polyvinyl alcohol, polyurethane, dextran, xanthan, hydroxypropyl cellulose, N-vinylpyrrolidone, N-vinyl lactam, N-vinyl butyrolactam, N-vinyl caprolactam, another vinyl compound having a polar pendant group, ethyl cellulose, hydroxyethyl cellulose, cellulose nitrate, cellulose acetate, cellulose acetate butyrate, cellulose acetate propionate, a natural or synthetic elastomer, a rubber, an acetal, nylon, polyester, styrene polybutadiene, acrylic resin, polyvinyl chloride, polyvinyl chloride acetate, polyvinylidene chloride, polycarbonate, a vinyl compound, or the like; or a copolymer obtained by combining any of those compounds.

In the case where the side chain capable of changing a functional group is a carboxyl group, a candidate material of a functional material can be prepared as a synthetic polymer complex by changing the carboxyl group with an ester, amide, halogenated alkanoyl, anhydride, aldehyde, alcohol, ketone, imide, lactam, or the like. Alternatively, in the case where the side chain is an amino group, the material can be prepared by changing the amino group with an amide, imine, enamine or nitro group, or the like.

The term “synthetic polymer construct” in the present invention refers to one present in a solid state in a solution at normal temperature. The material provided as a base to be coated with a polymer may be either an organic or inorganic material. Specific examples thereof include: synthetic polymers such as polystyrene, polyethylene, polypropyrene, polybutyrene, tetrafluoroethylene, polystyrene, an acrylic resin, nylon, polyester, polycarbonate, polyacrylamide, polyurethane, polyvinyl acetate, polyvinyl acetal, a rubber, a latex rubber, and silicone; natural polymers such as agarose, cellulose, cellulose acetate, chitin, chitosan, and alginate; inorganic materials such as hydroxyapatite, silica, alumina, titania, glass, mica, and carbon black; and metals such as stainless steel, titanium, and aluminum. The form of the carrier may be any form, and specific examples thereof include spheres, particles, a powder, a tip, a fiber, a flat membrane, a film, and a nonwoven fabric.

Examples of a method in which a polymer having a side chain capable of changing a functional group is coated on the surface of the synthetic polymer construct include: a method in which a monomer provided as a target candidate material is added when polymerizing a polymer to form a construct, to thereby prepare a coated layer; a method in which a polymer is adsorbed by physical adsorption; a chemical graft method, and a graft method using radiation, gamma rays, or electron beams.

The particles having a material to be evaluated at least on the surface thereof may preferably have a form of spheres, more preferably have a uniform particle size as much as possible. The particle size is preferably 2 μm to 100 μm, more preferably 5 μto 50 μm. If the particle size is more than 100 μm, there is a possibility of blocking the flow channel of a flow cytometer, which is not desirable.

According to the analysis method of the present invention, the biological substance composite particles are prepared by bringing the particles into contact with the biological substance to be evaluated for an interaction therewith, but a variety of methods, chosen depending on the applications of the material, can be used. For example, a material intended for a medical instrument for extracorporeal circulation or an artificial blood vessel can be subjected to a contact reaction under the condition in which the material particles are stirred at a certain flow rate and shear stress is added in a solution of the biological substance. In addition, for a material that is intended for a separating membrane such as a leukocyte-removing filter, a method of subjecting to a contact reaction by filtering a solution of the biological substance through a column filled with the material particles can be considered.

### EXAMPLES

Hereinafter, the present invention will be explained specifically by way of Examples, but the present invention is not limited thereto.

### Example 1

Preparation of Particles for Material to be Evaluated

Various polymers obtained by polymerization of polyhydroxyethyl methacrylate (pHEMA) or hydroxyethyl methacrylate (HEMA) with dimethylaminomethacrylate (DM) so as to be different in wt % were dissolved in ethanol heated to 40° C., to thereby prepare 2 wt % of a polymer solution. In the polymer solution (1 ml), 10 mg of polystyrene particles (Polybead™ Polystyrene Microspheres, PolyScience, USA) having a diameter of 10 μm were dispersed, to thereby prepare a suspension having a particle concentration of 1 wt %. The particle suspension was irradiated with ultrasound at 40° C. for 5 minutes. Subsequently, a container including the particle suspension was attached to a rotator (small rotation incubator RT 5, TAITEC, Japan), and heated to 40° C. in an incubator while being rotated at a speed of 3.4 m/min, followed by reaction for 55 minutes. After completion of the reaction, the particles were separated by centrifugation, and the obtained particles were washed with water and freeze-dried, to thereby prepare particles coated with a polymer obtained by copolymerization of pHEMA or HEMA with DM.

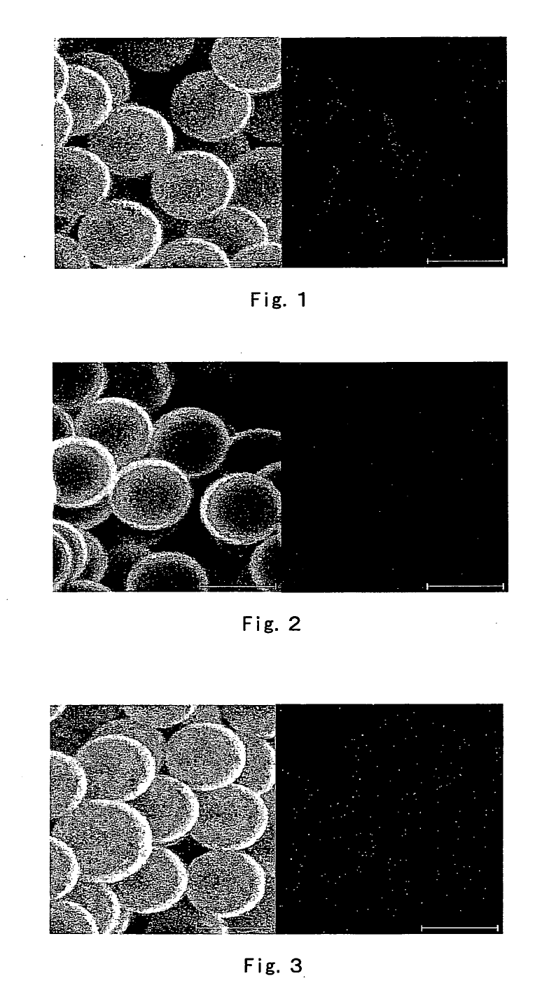

The particle surfaces were analyzed for particles coated with pHEMA as in Example 1, and for polystyrene particles serving as base material particles (Comparative Example 1) by using an X-ray photoelectron spectroscope (ESCA 5400, Perkin Elmer, Inc., USA). As a result, as shown in Table 1, it was confirmed that the particles of Comparative Example 1 contained about 99% of carbon atoms, while the particles of Example 1 had an element composition ratio approximate to that of carbon atoms and oxygen atoms composing polyhydroxyethylmethacrylate. The results for a copolymerized polymer of HEMA and DM are the same as above. Note that C % and 0% that are element compositions of the surface in FIG. 1 each represent an existence ratio of carbon atoms and oxygen atoms based on 100% of composition elements. In addition, the composition ratio of carbon and oxygen is determined by the chemical formula of HEMA and is shown as a reference.

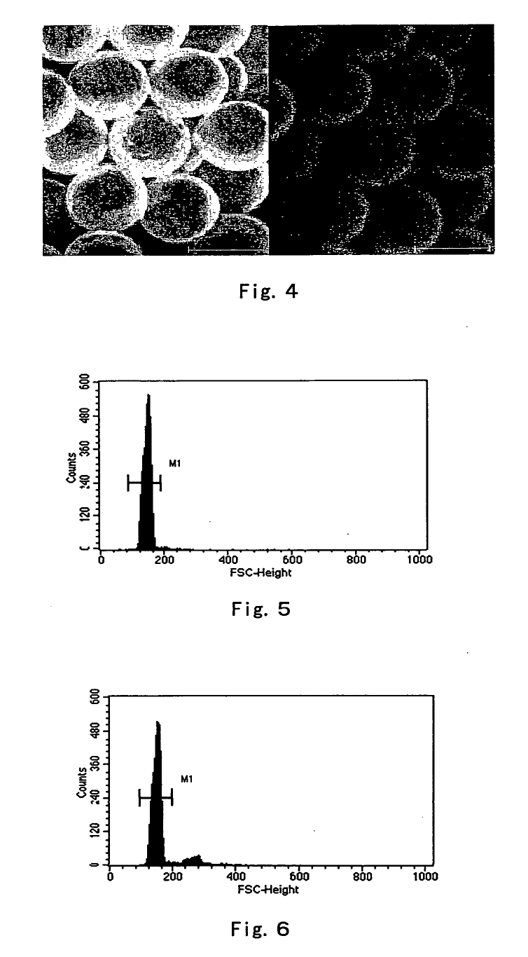

Moreover, the distribution state of the outermost surface is observed by using a time-of-flight secondary ion mass spectrometer (TRIFT II, Physical Electronics, Inc., USA). Note that, in FIGS. 1 to 4, each figure on the left shows the distribution of all ions detected by the aforementioned apparatus, and each figure on the right is an image showing the intensity distribution which is selected and plotted mass peaks based on ions derived from styrene or HEMA. The mass peaks based on ions derived from styrene that is a component of base material particles were detected in FIG. 1, but were hardly detected in FIG. 2. The mass peaks based on ions derived from HEMA were not detected in FIG. 3 (in the case of the base material particles of Comparative Example 1), but were detected as shown in FIG. 4 (in the case of the particles of Example 1). As a result, it was confirmed that the molecules were distributed on the entire surface of the particles. In addition, as shown in Table 2, in both cases of peak numbers 48 and 49, it was confirmed that the detection intensities of mass peaks based on ions derived from styrene that is a component of the base material particles was 5% or less. In the case of a polymer obtained by copolymerization of HEMA and DM, not only mass peaks derived from HEMA but also mass peaks derived from DM were confirmed.

TABLE 1

| | | Surface | |
|---|---|---|---|
| | | Element Composition | |
| Sample | | C % | O% |
| Comparative | Polystyrene particles | 98.5 | 1.3 |
| Example 1 | | | |
| Example 1 | Polystyrene particles + | 67.5 | 32.2 |
| | pHEMA-coated | | |
| Reference | HEMA composition | 66.7 | 33.3 |

TABLE 2

| | | Comparative | |
|---|---|---|---|
| | | Example 1 | Example 1 |
| | | Polystyrene | pHEMA-coated |
| Sample | | particles | particles |
| Mass peak intensity | 48 | 2994 | 82 |
| | 49 | 3463 | 178 |
| Total ion number | | 695190 | 1195959 |
| Mass peak intensity/ | 48 | 4307 | 69 |
| Total ion number | 49 | 4981 | 148 |
| Existence ratio of element | 48 | — | 1.6 |
| derived from Comparative | 49 | — | 3.0 |
| Example 1 (%) | | | |

The mass peaks derived from the polystyrene particles were strongly seen in the cases of mass numbers 48 and 49.

Further, in order to confirm the dispersibility of the particles of Comparative Example 1 and Example 1, each of suspensions obtained by re-dispersing each of the particles after drying in water was analyzed by using a flow cytometer (FACSCalibur, Becton, Dickinson and Company, USA). As a result, as shown in FIGS. 5 and 6 showing results of Comparative Example 1 and Example 1, respectively, the histograms of forward scattered light for those suspensions show almost the same monodispersion.

Fluorescent Labeling of Biological Substance

By using phosphate buffered saline (PBS), 10 mg/ml of human immunoglobulin G (IgG; SI; GMA, USA) solution was prepared, and 0.2 ml of the solution, 20 μl of 1M NaHCO₃, 27 μl of 10 mg/ml FITC (FITC-1; Molecular Probes, USA) dissolved in dimethyl sulfoxide were mixed, followed by reaction at room temperature for one hour while rotating a stirrer with the light shielded. Subsequently, a removing treatment was performed by adsorbing unreacted FITC to a resin, and then each of the absorbances of the final product was measured based on the fact that the labeled protein and FITC has absorptions in 280 nm and 494 nm, respectively. As a result, it was confirmed that an IgG1 molecule was bound with about seven FITCs. In addition, fluorescent labeling was performed on human fibrinogen (American Diagnostica, USA) by using Protein Labeling Kit of Alexa Fluor 647 (Molecular Probes, USA), the measurement similar to the above was performed based on the fact that the fluorescent dye has an absorption in 650 nm. As a result, it was confirmed that a molecule of fibrinogen had about eight bonds to the dyes.

Contact Reaction of Material Particles and Biological Substance and Measurement Using Flow Cytometer

Particles having each polymer on the surface thereof were suspended in a solution in which 0.2 mg/ml of fluorescent-labeled IgG and 0.2 mg/ml of fibrinogen are mixed in the presence of 1% goat serum (ICN Pharmaceuticals, USA), followed by a contact reaction. The flow cytometer used was FACSCaliber (BD, USA), and the software used for data collection and analysis was CELLQuest (BD, USA). In a forward and side scatter detection system and in a fluorescence detection system, in order to set the sensitivities of negative controls, fluorescent-unlabeled biological substance composite particles were used, and in order to control compensation, CaliBRITE Beads (BD, USA) provided as fluorescence standard particles were used to control voltage and amplifier gain. The particles to be measured using a flow cytometer were passed through a nylon mesh having a pore size of 100 μm in order to remove aggregates, followed by measurement.

Analysis Using Flow Cytometer

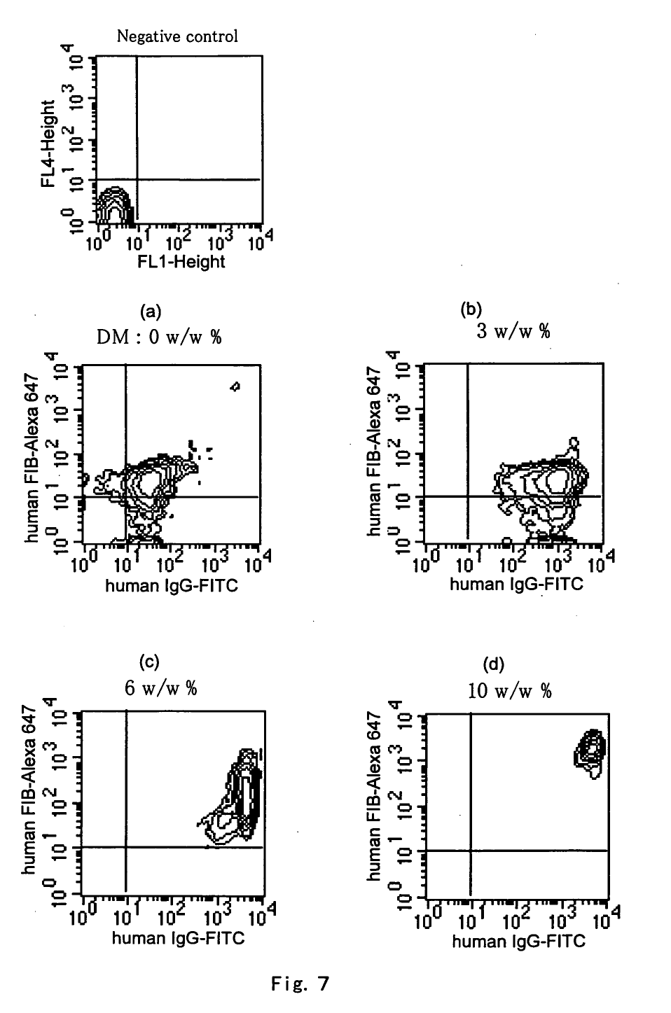

The upper most figure (negative control) in FIG. 7 shows a result of a negative control of the polymer particle that is not subjected to a contact reaction with IgG and fibrinogen. The other figures show results of copolymerized polymers of HEMA and DM analyzed using flow cytometer, in which values of DM wt % gradually increase (a value in (b) is larger than that in (a)). Each of the values of DM wt % to be used was 0, 3, 6, or 10%, respectively. It was shown that each material had a different adsorption state between fibrinogen and IgG.

### Example 2

Preparation of material particles Particles composed of copolymerized polymer of HEMA and DM were prepared in a manner similar to that of Example 1, and all the particles were confirmed to have been coated sufficiently. Moreover, uncoated particles were prepared by the same treatment as above with a solvent containing no polymer, and were used in Comparative Example 2.

Contact Reaction Between Material Particles and Biological Substance

A solution in which two kinds of protein, 0.2 mg/ml human fibrinogen (American Diagnostica, USA) and 20 mg/ml human albumin (KAKETSUKEN, Japan), were mixed was added with material particles, and the solution was mixed with shaking using a rotator (TAITEC, Japan) at a speed of 3.4 m/min at room temperature to perform a contact reaction.

Preparation of Biological Substance Composite Particles

After the contact reaction between the material particles and the biological substance, centrifugation was performed at 12,000 rpm for 5 minutes to remove unreacted biological substance. Subsequently, distilled water was added thereto, and the material particles were washed by centrifugation under the same conditions as above, to thereby yield biological substance composite particles.

Fluorescent Labeling of Biological Substance Composite Particles

Biological substance composite particles were subjected to an antigen-antibody reaction with an FITC-labeled goat anti-human fibrinogen antibody (ICN Pharmaceuticals, USA) and with a biotin-labeled goat anti-human albumin antibody (AMERICAN QUALEX Antibodies, USA) at 4° C. for 20 minutes, followed by a biotin-avidin reaction with phycoerythrin-labeled avidin (BD, USA), to thereby yield two-fluorescent-dye-labeled fibrinogen and albumin composite material particles.

Analysis Using Flow Cytometer

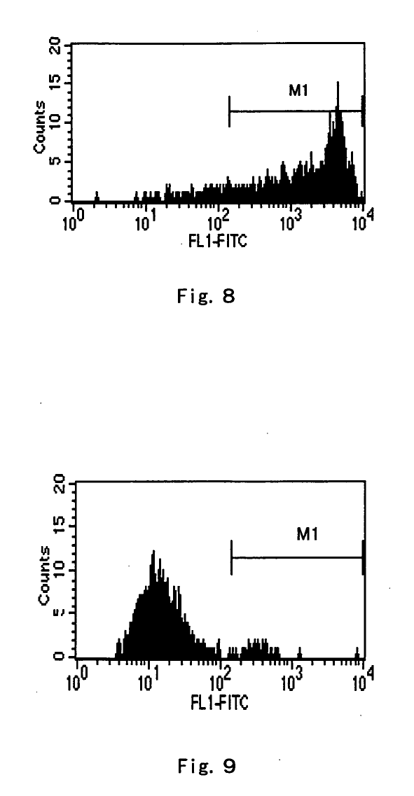

A flow cytometer set up in the same manner as in Example 1 was used to measure the adsorption of the material particle with fibrinogen or with albumin. FIGS. 8 and 9 are histograms showing the results for fibrinogen-adsorbed particles, which are represented by a fluorescence intensity of FITC labeled with fibrinogen indirectly. It can be found that the particle surfaces of Example 2 shown in FIG. 9 has difficulty in adsorbing fibrinogen compared with that of Comparative Example 2 shown in FIG. 8.

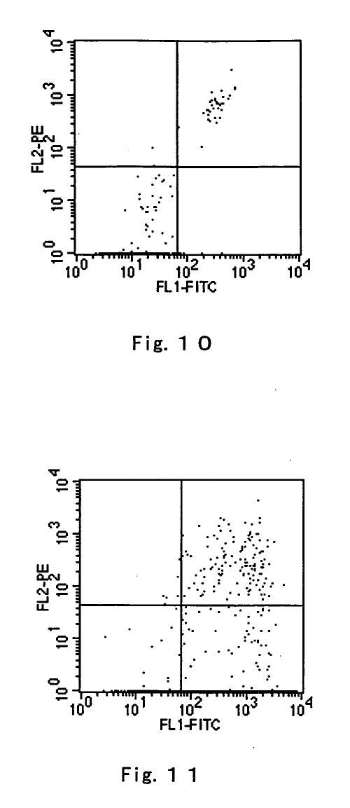

FIGS. 10 and 11 show two binding states with fibrinogen and with albumin of Comparative Example 2 and Example 2, respectively, which are represented by two-dimensional plots. As shown in those Figures, two items can be analyzed simultaneously.

### Example 3

Preparation of Material Particles to be Evaluated

Polymer particles were prepared by polymerization of HEMA with DM or with methyl methacrylate so as to be different in wt % in the same manner as in Example 1, and it was confirmed that all the particles were coated sufficiently.

Contact Reaction Between Material Particles and Biological Substance and Measurement Using Flow Cytometer

In Example 3, analysis of material particles was carried out using whole peripheral blood as a biological substance.

A contact reaction was performed by suspending the material particles in peripheral blood collected from a normal subject that is diluted ten fold with PBS. A part of the whole peripheral blood was taken and was passed through a nonwoven fabric filter of polyethylene terephthalate, to thereby prepare blood from which at least 99% platelets were removed. The resultant blood was subjected to the same procedure hereinafter as a negative control. Subsequently, Peridinin Chlorophyll Protein (PerCP)-labeled CD61 (BD, USA) provided as a platelet marker antibody and FITC-labeled PAC-1 antibody (BD, USA) provided as an activated platelet marker were added to the suspension of the material particles, and the adsorption of the material particles with platelets was measured using a flow cytometer set up in the same manner as in Example 1.

Analysis Using Flow Cytometer

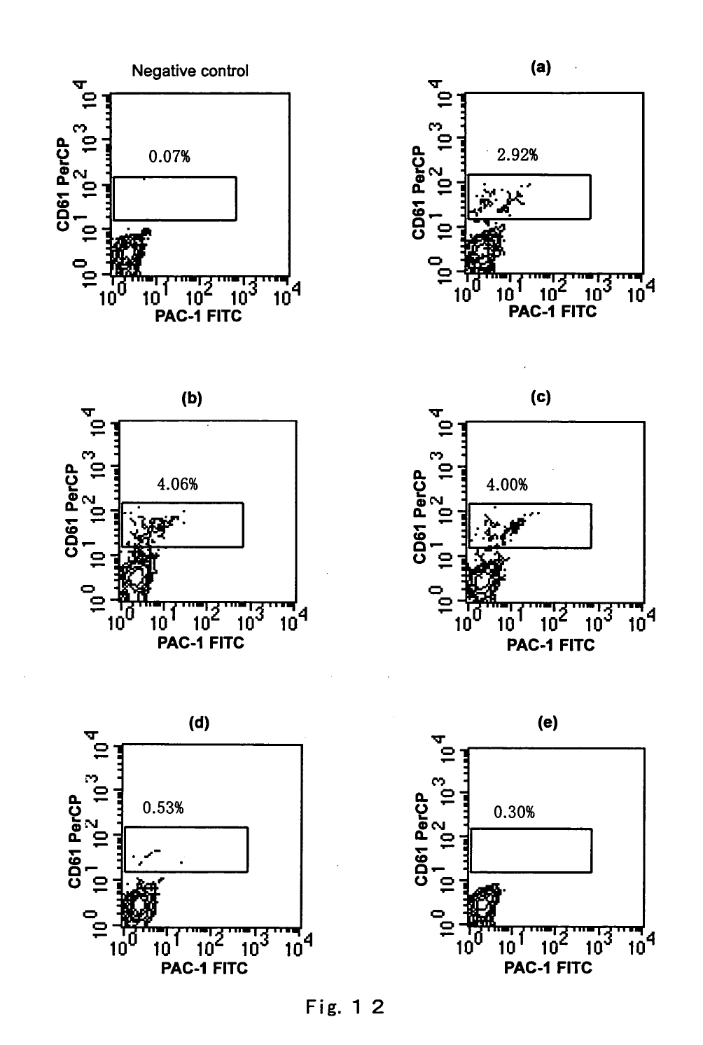

The upper left figure of FIG. 12 shows the result obtained by using platelet-removed blood. FIGS. 12(a) to (e) show a part of results obtained by analysis using a flow cytometer for various polymer material particles having different components or composition ratio. The adsorption properties between each of the materials and platelet can be compared and analyzed.

### Example 4

Fluorescent labeling was performed indirectly by the contact reaction of the material particles prepared in Example 3 with unreacted IgG and with fibrinogen, to thereby compare the materials.

Fluorescent Labeling of Biological Substance Composite Particles

A contact reaction between the material particles and the biological substance was performed by using IgG used in Example 1 and fluorescent-unlabeled fibrinogen as biological substances similarly to Example 2. After completion of the reaction, centrifugation was performed at 12,000 rpm for 5 minutes to remove unreacted biological substance. Subsequently, distilled water was added thereto, and the material particles were washed by centrifugation under the same conditions as above, to thereby yield biological substance composite particles. Then, an FITC-labeled goat anti-human fibrinogen antibody (ICN Pharmaceuticals, USA) and biotin-labeled goat anti-human IgG antibody (ICN Pharmaceuticals, USA) were added to 100 μl of the particle suspension to perform an antigen-antibody reaction at 4° C. for 20 minutes. After washing, a biotin-avidin reaction was performed with phycoerythrin (PE)-labeled avidin (BD, USA). In order to remove unreacted avidin, a washing step was performed, followed by analysis using a flow cytometer.

Analysis Using Flow Cytometer

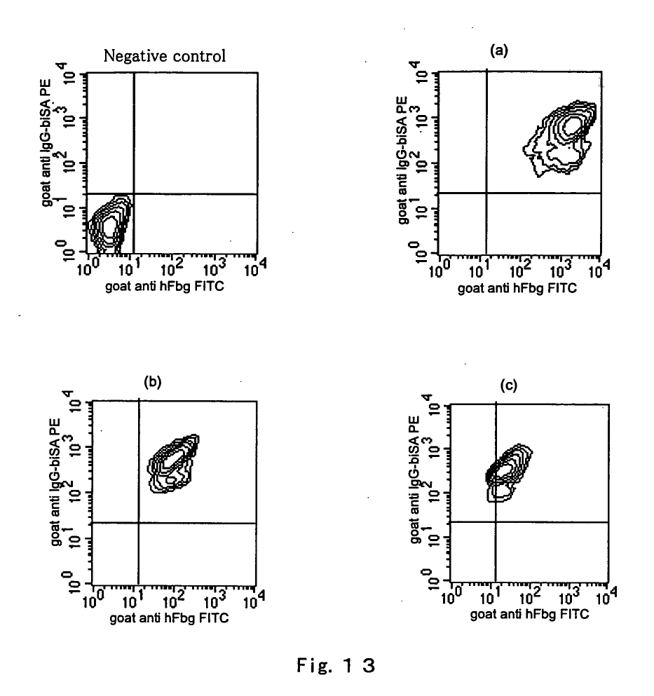

The upper left figure of FIG. 13 shows the result obtained by adding only antibody without the contact reaction with IgG and fibrinogen and by performing the following procedure in the same manner as above. FIGS. 13(a) to (c) show individual figures obtained by analysis using a flow cytometer for various polymer material particles having different components or composition ratios.

### INDUSTRIAL APPLICABILITY

According to the present invention, many materials can be easily and rapidly evaluated simultaneously using many indices under various conditions in a contact reaction. Therefore, the present invention can be used for selecting a material in development of a medical material and can greatly contribute to the development of medical instrument industries.

1. A method for analyzing an interaction between a material and a biological substance, characterized by analyzing an interaction between particles having a material to be evaluated at least on a surface thereof and a biological substance by using a flow cytometer.

2. The method according to claim 1, characterized by bringing the particle having a material to be evaluated at least on the surface into contact with the biological substance to prepare biological substance composite particles, and analyzing the biological substance composite particles by using a flow cytometer.

3. The method according to claim 2, characterized by carrying out fluorescent labeling of the biological substance directly or indirectly to thereby analyze biological substance composite particles by using a flow cytometer.

4. The method according to any one of claims 1 to 3, wherein the material to be evaluated is a medical material.

5. A medical material characterized in that the material is selected by using the analysis method of claim 4.

6. A medical material, characterized in that the material is selected from a plurality of medical materials to be evaluated so as to satisfy a predetermined criterion of the interaction between the biological substance and the material by using the analysis method of claim 4.

7. The method according to any one of claims 1 to 4, characterized by using polymer-coated particles obtained by coating the surface of particles composed of a base material with a polymer material by physical adsorption as the particles having a material to be evaluated at least on a surface thereof.

8. The method according to claim 7, wherein the properties of the surface of the polymer-coated particles satisfy at least one of the following conditions:
1) the element composition ratio on the surface is substantially equal to an element composition ratio of a polymer material based on X-ray photoelectron spectroscopy; and
2) the mass peak intensity derived from the base material particles element is 10% or less than that derived from a polymer material based on time-of-flight secondary ion mass spectrometry.

9. The method according to claim 7 or 8, wherein the polymer-coated particles are dispersed in a dispersion medium.

10. The method according to any one of claims 7 to 9, wherein the particle size of the base material particles of the polymer-coated particles is 0.1 ₙm or more and 100 ₙm or less.

11. The method according to any one of claims 7 to 10, wherein the sphericity of the base material particles of the polymer-coated particles is 0.50 or more.

12. The method according to any one of claims 7 to 11, wherein the average molecular weight of the polymer material is 1,000 or more and 3,000,000 or less.

13. The method according to any one of claims 7 to 12, characterized in that the polymer-coated particles are produced by a method comprising the steps of: suspending particles composed of a base material in a solution in which the polymer material is dissolved in a solvent; and applying physical vibration to the suspension.

14. The method according to claim 13, characterized in that the polymer-coated particles are produced by a method further comprising the steps of: after the step of applying physical vibration to the particles, washing the particles with a washing solution that is a poor solvent for the polymer material and base particles; and vacuum drying or freeze-drying the particles.

15. The method according to any one of claims 1 to 4, characterized in that the particles having the material to be evaluated at least on the surface thereof are produced by binding a synthetic polymer construct base material particles having a polymer with a side chain capable of changing a functional group at least on a surface thereof to a functional group of a compound having the functional group via the side chain.

16. The method according to claim 15, wherein the polymer having a side chain capable of changing a functional group is an acrylic or methacrylic polymer.
